(19) United States
(12) Patent Application Publication
(10) Pub. No.: US 2013/0023674 A1
(43) Pub. Date: Jan. 24, 2013

(54) Lactide Production from Thermal Depolymerization of PLA with applications to Production of PLA or other bioproducts

(71) Applicants: Ramani Narayan, Okemos, MI (US); Wei-Min Wu, Okemos, MI (US); Craig S. Criddle, Redwood City, CA (US)

(72) Inventors: Ramani Narayan, Okemos, MI (US); Wei-Min Wu, Okemos, MI (US); Craig S. Criddle, Redwood City, CA (US)

(21) Appl. No.: 13/421,780

(22) Filed: Mar. 15, 2012

Related U.S. Application Data
(60) Provisional application No. 61/465,143, filed on Mar. 15, 2011.

(51) Int. Cl. C07D 319/12

(52) U.S. Cl. 549/274

(57) ABSTRACT

Methods and systems are disclosed for producing lactide, which can be used for PLA production or other valuable bioproducts. PLA is heated to undergo thermal depolymerization to recover lactide. The lactide can be used for PLA production or other valuable bioproducts.

### CROSS-REFERENCE TO RELATED APPLICATIONS

This application claims priority from U.S. Provisional Patent Application 61/465143 filed Mar. 15, 2011, which is incorporated herein by reference.

### FIELD OF THE INVENTION

The present invention relates generally to methods and systems for producing lactide, which can be used for PLA production or other valuable bioproducts. More specifically, the present invention provides techniques for depolymerization with heat to recover lactide, which can be used for PLA production or other valuable bioproducts.

### BACKGROUND OF THE INVENTION

Current petroleum-based plastics have several drawbacks. In addition to being produced from non-renewable petroleum resources, they do not appreciably biodegrade if they end up littered in the ocean. These plastics build up and choke the marine environment, creating a significant plastic ocean debris problem. Motivated by these and other drawbacks, it would be desirable to replace these petroleum-based plastics with new bioplastics that will biodegrade if they are littered, are not made from non-renewable resources, and are sustainable (i.e., can be recycled to complete the cradle-to-cradle cycle).

Unlike petroleum-based plastics which have slow rates of degradation, certain bioplastics can degrade rapidly, as fast as weeks or months, and thus do not accumulate over time if littered in the oceans. A major obstacle to their wider use is cost. A major component of the production cost is the cost of harvesting and transporting feed-stocks to a production facility. This process uses the existing collection systems for organic wastes and landfill gas as feed-stocks.

Current production of bioplastics is in its infancy. Bioplastics are produced in the US, Europe, Asia and South America. All of the current production of bioplastics uses some form of food crop as feedstock. The use of food crops as the starting material for bioplastics is expensive because it is energy-intensive, demands the use of water, fertilizers and pesticides, and in some cases, raises the consumer costs of food due to the competition.

### SUMMARY OF THE INVENTION

To address these issues, and the need to reduce organic waste going to landfills, the present invention provides methods to make bioplastics from waste rather than from food or petroleum.

The current method avoids the cost of harvesting and transporting feed-stocks to a production facility by using feed-stocks derived from organic waste streams already present in municipal solid wastes that are already collected in centralized facilities.

The invention also includes a novel process to use waste as feedstock to make the bioplastics PHB, PHA and PLA. The process establishes three bioplastic production lines from existing organic waste streams: One line yields polyhydroxybutyrate (PHB) from waste-derived biogas methane. Another yields polyhydroxyalkanoate (PHA) biopolymers from C5 xylose sugars hydrolyzed from municipal solid waste. A third line yields polylactic acid (PLA) biopolymers from C6 glucose sugars hydrolyzed from municipal solid waste.

The invention is a novel process to make three bioplastics in the most environmentally friendly manner. A significant novel feature is the use of heat, not chemicals, for the extraction of PHB and PHA. The current state of the art uses chlorinated solvents.

The invention establishes new cradle-to-cradle processes, so that each of the bioplastics is recycled back into a downstream location of the processes. A new and not obvious feature is that this new recycling creates a more efficient usage of the recycled materials because they are introduced into the manufacturing processes significantly downstream to where recycling is done presently. For PHB and PHA, the recycled polymer is introduced at the rotary drum heater. For the PLA, the recycled polymer is heated and depolymerized to lactide, and then introduced to the lactide reactor. This is a significant improvement to the current process of turning PLA to lactic acid, which is then introduced to the process.

Petroleum plastics can take a thousand years to biodegrade into chemical constituents. The three bioplastics, polyhydroxybutyrate (PHB), polyhydroxyalkanoates (PHA), and Polylactide (polylactic acid, PLA) all can be biodegraded into their chemical constituents much more rapidly, as fast as weeks or months, instead of a thousand years. The invention also establishes new cradle-to-cradle processes, so that each of the bioplastics is recycled back into the processes. The invention also includes a novel process to use waste as feedstock to make the bioplastics.

This invention establishes cradle-to-cradle processes for polyester bioplastics—polylactic acid (PLA) and polyhydroxyalkanoic acids (PHAs). Typically these biopolymers are made from crop feed-stocks that require harvesting and transport to a biopolymer production facility. These harvest and transport costs are avoided by taking advantage of transportation systems already in place for the collection of municipal solid waste.

Organic waste streams are often viewed as liabilities. The invention provides process lines in which these streams becomes feed-stocks for the production of valuable biopolymers. One important stream is biogas methane from landfills, wastewater treatment plants, and the dairy industry. If released to the atmosphere, the methane generated at these facilities can contribute to climate change because methane is a strong greenhouse gas 21 times more potent than carbon dioxide. The waste methane can be used to make polyhydroxybutyrate (PHB). Another important waste stream is the solid organic fraction of municipal solid waste (MSW). There is already an infrastructure to collect MSW and bring it to landfills. In California, the MSW passes through a sorting facility called a Materials Recovery Facility (MRF) prior to land-filling. At the MRF, any recyclable metal, can or bottle is removed for recycling. At the end of the process, what is left is called the MRF residue. The MRF residue has a large percentage of organic material. This organic material is a form of cellulosic biomass. The cellulosic biomass can be treated to release sugars from the cellulose and hemi-cellulose, which comprise about two thirds of the organics. About one third consists of lignin, a carbonaceous material that is not readily converted to sugars, but can be used as a colorant to darken the bioplastic products, or burned for energy recovery.

There is a large research effort in the US to find an economical method to convert cellulosic wastes into ethanol. Prior to the step to make ethanol, glucose and xylose (C6 and C5) sugars are the intermediate products. Instead of using the sugars to make ethanol, it is also possible to use the methods and sugars under the ethanol research to make bioplastics, because the sugars are good food sources for many bioplastics-producing bacteria. One of the cellulosic ethanol technologies developed (dilute acid) is incorporated in this invention as a pre-treatment method for the MRF residues.

The term biorefinery has been used in several ways in the past for industrial production of fuels or products from non-petroleum feed stocks. This invention provides a biorefinery system and method that primarily produces bioplastics from waste. Other value added products could also be produced by the invention, including energy, biofuels and biochemicals.

The invention will have application at landfills where municipal solid waste is collected and other organic waste collection facilities. The PHB/PHA and PLA biopolymers that are produced are of value in a wide range of applications.

### BRIEF DESCRIPTION OF THE DRAWINGS

FIG. 1: Schematic diagram of system for producing PHB from waste methane

FIG. 2: Schematic diagram of system for producing PHA from MRF residues

FIG. 3: Schematic diagram of system for producing PLA from MRF residues PFD

### DETAILED DESCRIPTION

Embodiments of the invention provide process lines to make three bioplastics in an environmentally friendly manner. One process line produces PHB from waste-derived methane. Another process line produces PHA from C5 xylose sugars hydrolyzed from MSW. A third process line produces PLA from C6 glucose sugars hydrolyzed from MSW.

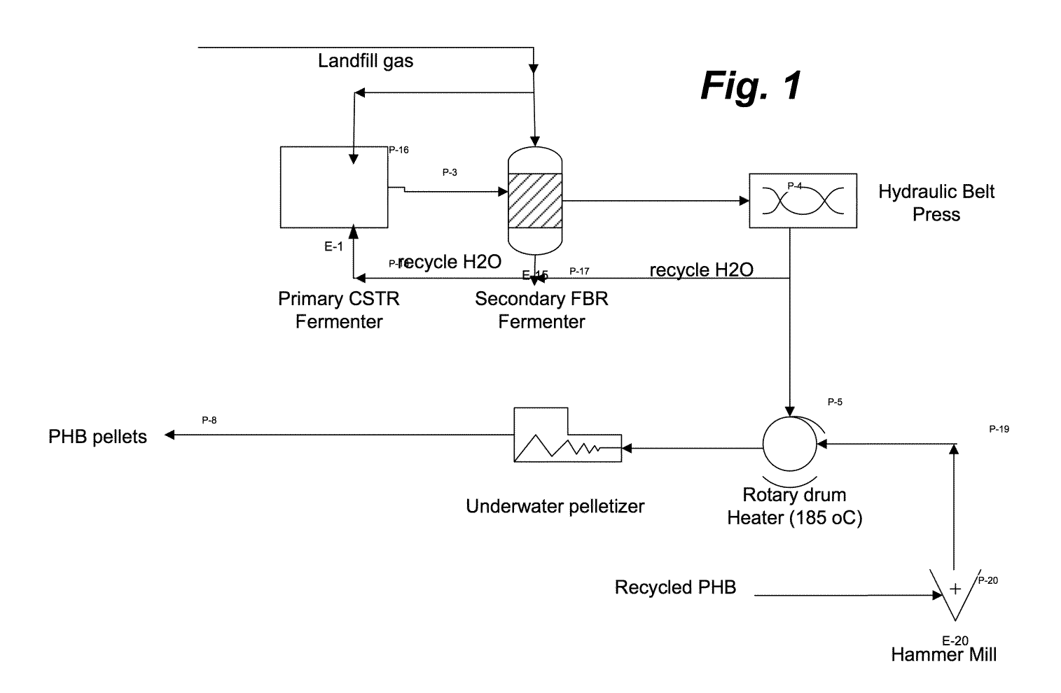

The first process line, illustrated in FIG. 1, produces PHB from waste-derived methane (landfill gas) and recycled PHB. The process incorporates sustainable cradle-to-cradle aspects that use recovered PHB as feedstock. For the recycling steps, the PHB is first broken into small pieces by a hammer mill, and then melted in a rotary drum heater, or equivalent, to separate impurities and send the material to a pelletizer where new PHB product resin pellets can be formed. The PHB process from methane includes the methane landfill gas feed system, a primary fermenter to multiply methanotrophic bacteria cells, followed by a secondary fermenter to grow PHB under nutrient-starved conditions.

The resultant mixture is sent to a hydraulic belt press with centrifugal pumps to remove much of the water. The pumps act to partially homogenize the cells to break some open. This is followed by a rotary drum heater to melt the PHB from the biomass mixture. Most biomass cells that are still intact will break open by steam explosion in the heater.

The recycled PHB is also sent to the heater after it is broken into pieces by the hammer mill. The molten PHB is sent to an underwater pelletizer to form PHB resin pellets. The same heater is used for the PHB-biomass mixture and the recycled PHB particles. In another similar process, a dedicated heater and pelletizer could be used just for the recycled PHB.

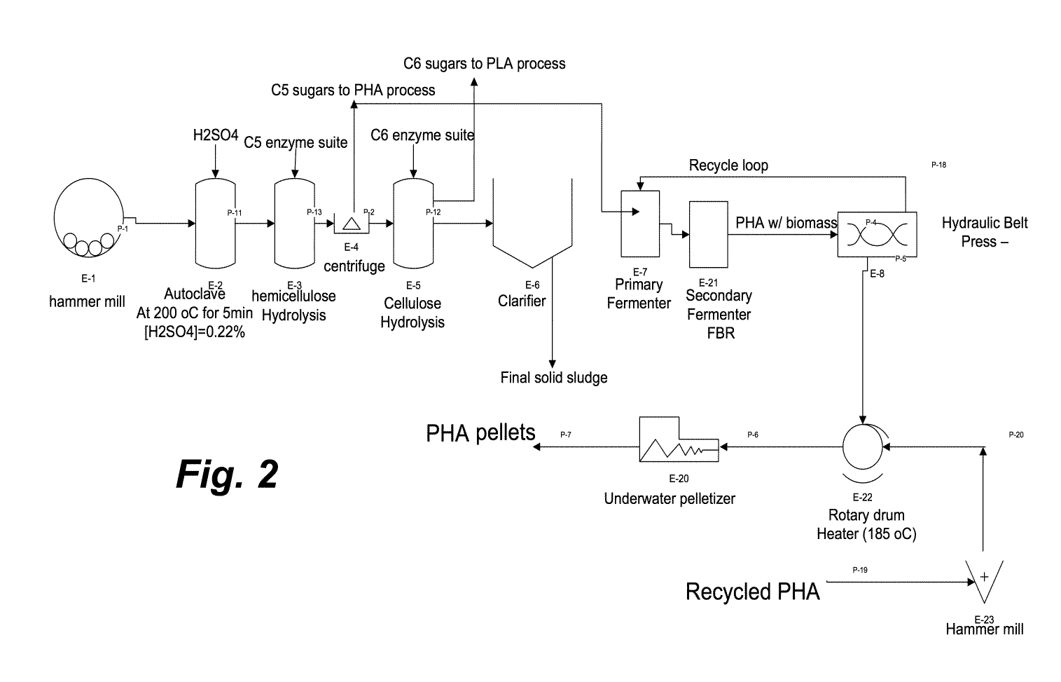

The second process line, illustrated in FIG. 2, produces medium-chain-length PHA from MSW residues and recycled PHA.

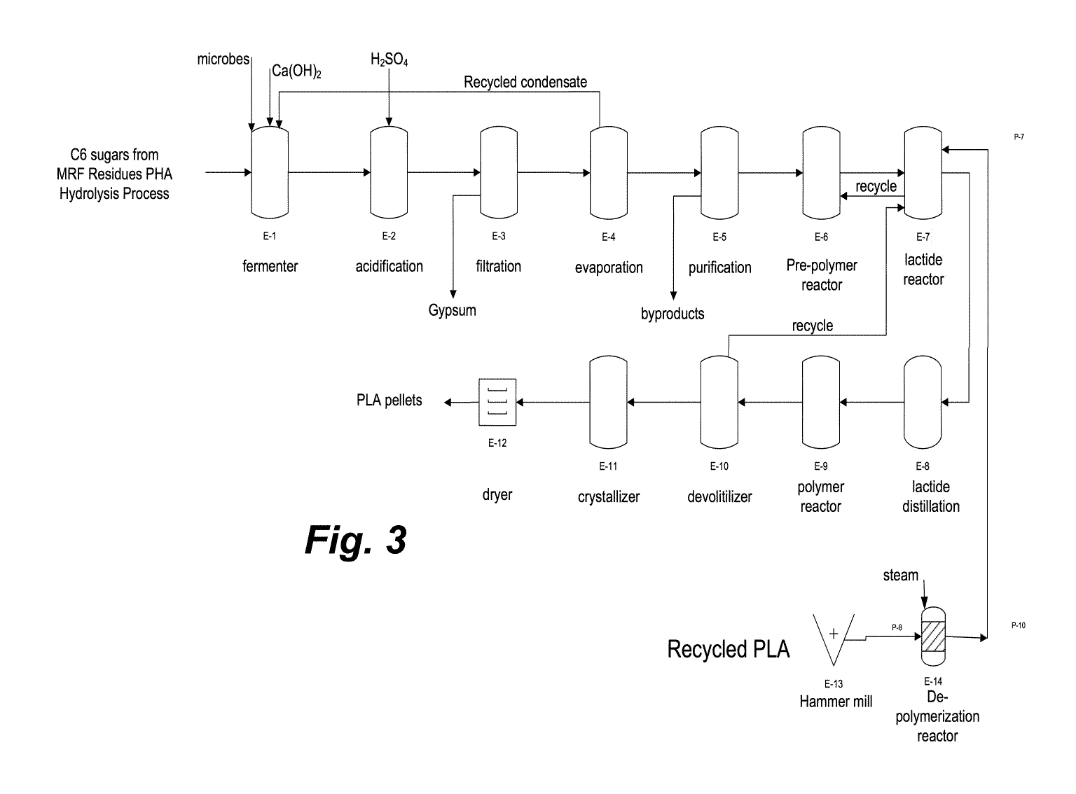

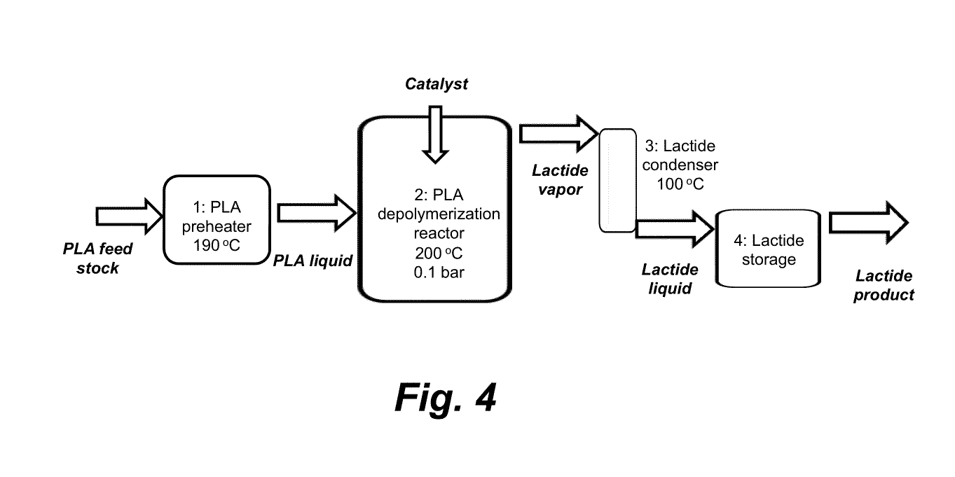

Pre-treatment of MSW residues is performed by treatment with dilute acid followed by enzymes. First, a hammer mill (upper left) is used to reduce the size of MSW particles fed to the line. Next, these particles are made into a slurry by adding water to the pulverized MSW as it enters the autoclave. In the autoclave, H₂SO₄ is added to make the dilute concentration of 0.22%, and then treated by the autoclave at 200° C. for 5 min to begin producing C5 xylose sugars. Next, a C5 enzyme mixture is added to a heated hemi-cellulose hydrolysis tank to further extract C5 xylose sugars. The mixture is sent to a centrifuge to separate the C5 xylose sugars for PHA fermentation. The C5 xylose sugars are sent from the centrifuge to the primary fermenter. Meanwhile, the material that remains from the centrifuge (i.e., unreacted MSW material depleted of hemi-cellulose) is sent to the heated cellulose hydrolysis tank, where a C6 enzyme suite is added to produce C6 glucose sugars. The C6 glucose sugars are sent to the PLA process line (see FIG. 3 and related description below). The waste from the cellulose hydrolysis tank is sent to a dedicated hydraulic belt press, and the recycled water from the dedicated hydraulic belt press is sent back to the autoclave.

The primary fermenter receives the C5 xylose sugars and nutrients so that the bacteria (Pseudomonas oleovorans but not limited to it) will multiply. The primary fermenter can be designed so that growth and decay of cells are balanced so that there is a relatively constant optimal concentration of bacterial cells. As cells are continuously removed to seed the secondary fermenter, more substrate and nutrients are continuously added to maintain the cell concentration. In the secondary fermenter, nutrients are limited so that PHA is grown by the cells, while C5 sugars are added as substrate to grow the PHA. The resulting mixture is sent, by centrifugal pumps, to a hydraulic belt press to remove much of the water. This is followed by a rotary drum heater to melt the PHA from the biomass mixture. The molten PHA is sent to an underwater pelletizer to form PHA resin pellets. For the recycling steps, the PHA is first broken into small pieces by a hammer mill, and then melted in the rotary drum heater, or equivalent, to separate impurities and send the material to a pelletizer where new PHA product resin pellets can be formed. A dedicated heater and pelletizer could be used if the recycled PHA (or PHB for the first process line) becomes too large for the existing biorefinery size.

The third process line is to produce PLA from MSW residues and recycled PLA. Part of this process is based on the processes used to make PLA from corn, published by Vink (2010). In this process, however, the C6 glucose sugars are made from MSW residues, not from corn, so Vink's method is adapted to account for the feed stocks, and the recycling of PLA. The pretreatment of MSW residues is listed in the PHA from MSW residue process line listed above in relation to FIG. 2. This PLA process line, in contrast to Vink's description of the Natureworks LLC process, incorporates the cradle-to-cradle recycling of PLA, as well as using waste as feed stock rather than corn. The recycling is done by a hammer mill to make small PLA particles, followed by a PLA heated depolymerization reactor. The resultant intermediate product is a liquid lactide, which is sent to the lactide reactor within the process.

The C6 glucose sugars from the MRF residues PHA hydrolysis (FIG. 2) are fed to the fermenter, as are nutrients. The fermenter turns the sugars into lactic acid, and Ca(OH)2 is added to adjust the pH. The mixture is then acidified with H2SO4, which produces gypsum. Next, the solid gypsum is filtered from the liquid. Water is then evaporated from the liquid, condensed, and put back into the fermenter. The slurry from the evaporator is next purified by a membrane purifier, and the resulting mixture is sent to the pre-polymer reactor, depolymerizing to lactide monomer. Next, the lactide reactor produces lactide cyclic dimer with feed from the recycled PLA, and effluent from the pre-polymer reactor. The mixture is then distilled to purify the lactide cyclic dimer, which is sent to the polymer reactor, which produces the Polylactide (PLA—polylactic acid). The PLA is then separated from all by-products by a devolitizer, using a twin screw extruder at 220° C. and 5 mmHg, along with a nitrogen gas blanket. The polymer is then sent to a crystallizer for polishing, finished by a dryer to remove the trace amounts of moisture.

Methods for Lactide Production from Thermal PLA Depolymerization

In general, the present invention provides a method for the production of lactide directly from recycled PLA wastes using thermal depolymerization process. The novel process is illustrated in FIG. 4. PLA feed stock is fed into a PLA preheater 1 where temperature is maintained at around 190° C. (above PLA melting temperature 173-178° C.). After melting, PLA liquid is pumped into PLA depolymerization reactor 2. The temperature of the reactor 2 is maintained at around 200° C. and the reactor headspace is maintained under near vacuum condition (0.1 bar). Catalyst likes Ti(II) ethylhexanoate (but not limited to it) is added in the reactor 2 to allow a reversed reaction (PLA→Lactide) occur with continuous depolymerization of PLA to lactide. The lactide vapor produced from the reaction is continuously removed from the reactor 2 by vacuum extraction and then condensed as liquid lactide in lactide condenser 3. The temperature of the condenser is maintained at around 100° C. which is above the melting point of 96° C. of L-lactide. The condensed lactide flows into lactide storage tank 4 and collected as product. The impurity solids such as petroleum based plastics and others, if mixed in PLA feed stock, cannot form vapor in headspace and remains in the reactor 2 and are removed periodically during reactor maintenance.

This process can be operated with either continuous feeding mode or fed batch mode. With continuous mode, PLA feed stock is fed to the system continuously via the preheater 1 and then PLA liquid is fed to the reactor 2. With fed batch mode, the PLA liquid is fed to the reactor 2 till ½-⅓ reactor volume and the feed is stopped. Depolymerization of PLA is then started till conversion of 80-90% of PLA to lactide. Subsequently, the reactor 2 is refilled with the liquid PLA for the next reaction cycle.

Experimental Results

Experiments were performed to prove the methods. PLA resins, commercial PLA products and PLA-PEcoflex™ (poly(butylene adipate-co-terephthalate)) blends from NatureWorks were tested in fed batch mode. The feed stock started melting at 170° C. and depolymerization occurred at 195-200° C. in the presence of Ti(II) catalyst (0.05-0.1%, w/w). The conversion efficiencies from PLA to lactide were greater than 91-95% (w/w) and the products were mainly L-lactide, as shown in the table below.

| Sample | % L- | % Meso- | % D- | Optical | |
|---|---|---|---|---|---|
| # | Lactide | Lactide | Lactide | rotation | Note |
| 1 | 93.49 | 0.1530 | 6.70 | −225.32 | Nature Works |
| | | | | | PLA Resin |
| 2 | 93.49 | 0.1550 | 6.60 | −225.61 | Nature Works |
| | | | | | PLA Resin |
| 3 | 90.43 | 0.1536 | 5.80 | −211.35 | PLA 50% |
| | | | | | Ecoflex 50% |
| 4 | 88.17 | 0.1540 | 5.89 | −219.21 | PLA 90% |
| | | | | | Ecoflex 10% |
| 5 | 91.79 | 0.1528 | 6.20 | −219.92 | Nature Works cup |

1. A method for lactide production from PLA feedstock, the method comprising:
melting the PLA feedstock in a heater to produce PLA liquid;
heating the PLA liquid with a catalyst to depolymerize the PLA liquid and produce lactide vapor under vacuum atmosphere;
condensing the lactide vapor to produce liquid lactide.
